(19) United States
(12) Patent Application Publication
(10) Pub. No.: US 2007/0235043 A1
(43) Pub. Date: Oct. 11, 2007

(54) PRESBYOPIA TREATMENT BY WEAKENING THE ZONULA

(76) Inventor: Georges Baikoff, Marseille (FR)

Correspondence Address: SHELDON MAK ROSE & ANDERSON PC, 100 East Corson Street, Third Floor, PASADENA, CA, 91103-3842, US

(21) Appl. No.: 11/279,176

(22) Filed: Apr. 10, 2006

(51) Int. Cl. A61F 9/008

(52) U.S. Cl. 128/898; 606/4

(57) ABSTRACT

A method for treating presbyopia in the eye of a patient includes the step of applying energy to the zonula to weaken the zonula tension or the tension exerted on the zonula sufficiently to reverse the symptoms of presbyopia in the eye. In one example of the method, the energy applied to the zonula is electromagnetic energy, such as provided by a suitable laser.

### BACKGROUND OF THE INVENTION

Presbyopia is a loss of the eye's power to accommodate which takes place when a person ages, that is to say, the eye loses its ability to increase its refractive power. FIG. 1 is a diagrammatic representation of an eye 1 showing the lens 2 enclosed in the lens sac 3 and suspended from the ciliary body 4 by means of the zonule 5. The ciliary body 4 lines the internal surface of the sclera 6 about a ring located on the average at a latitude distance by 2 to 3 mm from the limbus 7, measured along the optical axis.

New methods for treating presbyopia have recently been disclosed wherein implant elements are disposed within small tunnels formed within the sclera of the patient's eyes. Once disposed in the scleral tunnels, the implants act on the sclera to enhance the ability of the patient's lens to contract, thereby diminishing the presbyopia condition. Examples of such new methods are disclosed, for example, in my previously-issued patents, U.S. Pat. Nos. 6,682,560 and 6,692,524, the entireties of which are incorporated herein by this reference.

FIG. 2 illustrates one of these new methods. An implant element 8 is surgically disposed within an incision tunnel 9 formed in the sclera 6 opposite the zonule 5. The tunnel 9 is disposed at a depth of about 600μ below the surface of the sclera 6. The tunnel 9 is typically about 8 mm long and about 3 mm wide. The implant element 8 favorably effects the adjustment of the lens shape by the sclera 6 and the zonule 7 to assist the residual accommodation, and therefore minimize the effects of presbyopia.

As promising as these new methods are for treating presbyopia, they suffer from the need for subjecting the patient to delicate eye surgery. Accordingly, there is a need for a method for treating presbyopia which avoids the necessity of eye surgery.

### SUMMARY OF THE INVENTION

The invention satisfies this need. The invention is a method for treating presbyopia in a patient comprising the step of applying energy to the zonula of the affected eye or eyes of the patient to weaken the zonula tension or the tension exerted on the zonula sufficiently to reverse the symptoms of presbyopia in the patient.

### DRAWINGS

These and other features, aspects and advantages of the present invention will become better understood with reference to the following description, appended claims and accompanying drawings where:

FIG. 1 is a schematic view in cross-section of an eye;

FIG. 2 is a fragmentary cross-sectional view of an eye in which is implanted a corrective element for the treatment of presbyopia; and

FIG. 3 is a schematic perspective view showing in detail the interior zonular fiber system in a human eye.

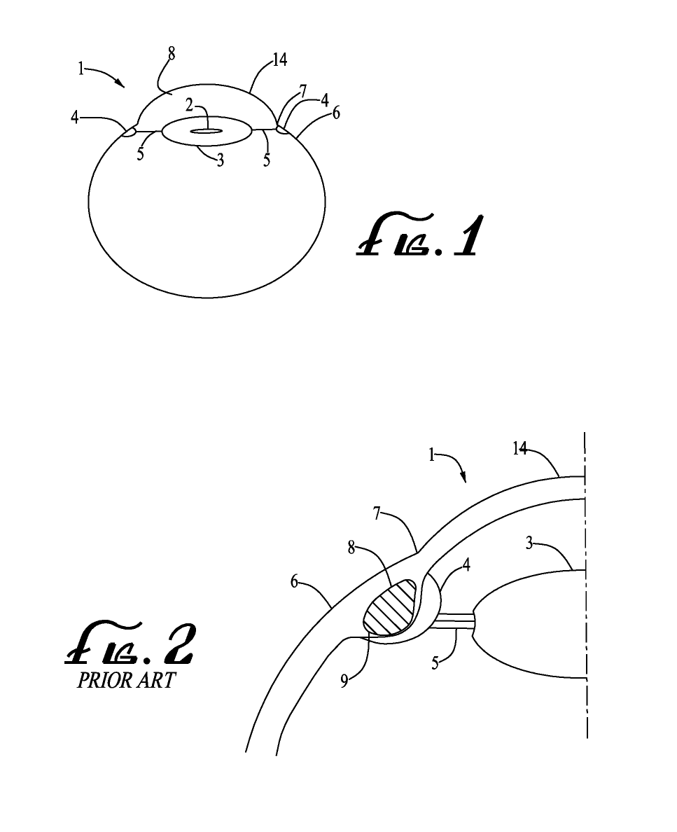

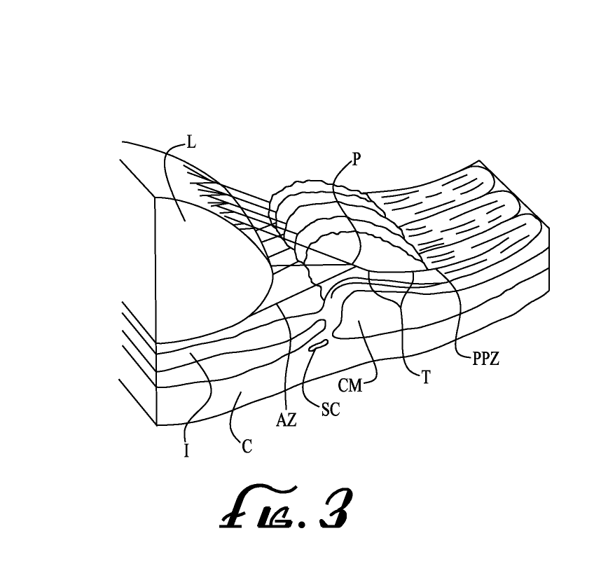

### DETAILED DESCRIPTION

The following discussion describes in detail one embodiment of the invention and several variations of that embodiment. This discussion should not be construed, however, as limiting the invention to those particular embodiments. Practitioners skilled in the art will recognize numerous other embodiments as well.

The invention is a method for treating presbyopia comprising the step of applying energy to the zonula of the affected eye or eyes of a patient to weaken the zonula tension or the tension exerted on the zonula sufficiently to reverse the symptoms of presbyopia in the patient.

Applying energy to the zonula to weaken the zonula tension results in a slackening of the posterior zonula tendon (or the posterior zonula) which creates a forward and inward thrust of the zonular plexus and facilitates the reduction of the crystalline lens diameter at the time of the contraction of the ciliary body, thereby reducing the symptoms of presbyopia.

The application of energy can be done either directly on the zonula or on the insertion zones of the zonula in order to move the position of the insertion, or to reduce tension on the zonula.

Typically, the application of energy to the zonula is accomplished by applying electromagnetic energy to the zonula. The applying of electromagnetic energy to the zonula is preferably carried out using a laser, since the energy emitted from a laser can be carefully controlled with respect to direction, wavelength and intensity.

The electromagnetic energy can be applied to the zonula either from the outside of the eye or from within the interior of the eye using, for example, an endoscope.

A simple method of applying electromagnetic energy to the zonula is by use of a three-mirrored lens.

Preferably, the application of electromagnetic energy to the zonula is carried out after pressing down on the sclera in order to reach the pars plana zone and to carry out the endoscopic laser section of the zonula itself or the zonula tendon, with or without applying the laser beam on the pars plana.

Lasers capable of properly applying electromagnetic energy to the zonula are well known in the field and include lasers having operating parameters of five μJ, 1 kHz, 500 fs, and 1.06 μm and 5 μJ, 1 kHz, 150 fs, and 775 nm. With either such laser, the laser beam can be focused through a lens, such as a 0.5 NA aspheric lens corrective for wave front flatness, such as described in “High Precision Subsurface Photodisruption in Human Sclera,” authored by Sacks et al. and published in Journal of Biomedical Optics, July 2002. Both such lasers can be used as described in the Sacks article to impart fentosecond pulses to the zonula.

In FIG. 3, the anterior zonular apparatus is depicted in detail. The lens L is suspended in a sac or capsule via the anterior zonules AZ, which converge at the zonular plexis P (sometimes also called the zonular fork). Posteriorly of the zonular plexis P, the posterior zonular fibers PPZ extend as suspensory ligament in the area of the pars plana, wherein T designates the tension fiber system in the area of the ciliary valleys. Also denoted in FIG. 3 are the iris I, cornea C, Schlemm canal SC and the ciliary muscle CM.

At the zonular plexis P, the zonular apparatus is connected to the ciliary process or the ciliary valleys via the tension fiber system T, and is therefore fixed to the ciliary body.

In the invention, the electromagnetic radiation can be directed to sever some of the zonular fibers AZ between the ciliary body and the crystalline lens. Alternatively, the electromagnetic radiation can be directed to cut zonular fibers T between the insertion at the pars plana and the ciliary process. In a third alternative, electromagnetic radiation can be directed to weaken the zonular tendon PPZ which rests against the inner sides of the sclera behind the ciliary processes.

Having thus described the invention, it should be apparent that numerous structural modifications and adaptations may be resorted to without departing from the scope and fair meaning of the instant invention as set forth hereinabove and as described hereinbelow by the claims.

What is claimed is:

1. A method for treating presbyopia in the eye of a patient, the eye having a crystalline lens supported by zonula, tension being exerted on the zonula to create zonula tension, the method comprising the step of applying energy to the zonula to weaken the zonula tension or the tension exerted on the zonula sufficiently to reverse the symptoms of presbyopia in the eye.

2. The method of claim 1 wherein the step of applying energy to the zonula comprises the step of applying electromagnetic energy to the zonula.

3. The method of claim 2 wherein the electromagnetic radiation is directed to sever some of the zonular fibers between the ciliary body and the crystalline lens.

4. The method of claim 2 wherein the electromagnetic radiation is directed to cut zonular fibers between the insertion at the pars plana and the ciliary process.

5. The method of claim 2 wherein the electromagnetic radiation is directed to weaken the zonular tendon which rests between the inner sides of the sclera behind the ciliary process.

6. The method of claim 2 wherein the electromagnetic radiation is applied directly on the zonula.

7. The method of claim 2 wherein the zonula are supported by insertion zones and wherein the electromagnetic radiation is supplied to the insertion zones.
